(19) United States
(12) Patent Application Publication
(10) Pub. No.: US 2013/0133778 A1
(43) Pub. Date: May 30, 2013

(54) INCUBATING CONTAINER AND SAMPLE INJECTION METHOD THEREFOR

(71) Applicants: Sang Hyun YI, Suwon (KR); Dong Woo LEE, Suwon (KR); Jeong Suong YANG, Yongin (KR); Bo Sung KU, Suwon (KR)

(72) Inventors: Sang Hyun YI, Suwon (KR); Dong Woo LEE, Suwon (KR); Jeong Suong YANG, Yongin (KR); Bo Sung KU, Suwon (KR)

(21) Appl. No.: 13/588,800

(22) Filed: Aug. 17, 2012

(30) Foreign Application Priority Data
Nov. 28, 2011 (KR) 10-2011-0124985

(51) Int. Cl. C12M 1/00; B65B 3/04; C12M 1/12

(52) U.S. Cl. 141/1; 435/297.1; 435/303.1

(57) ABSTRACT

There are provided an incubating container capable of preventing bubble generation within a cell incubating container when a sample is injected into the container, and a sample injection method therefor. The incubating container includes: a plurality of wells to be filled with a sample through an injection of the sample; and at least one bubble discharge hole formed as a hole penetrating through a bottom of each of the plurality of wells, and discharging, to the outside, bubbles generated when the sample is injected into the plurality of wells.

### CROSS-REFERENCE TO RELATED APPLICATIONS

This application claims the priority of Korean Patent Application No. 10-2011-0124985 filed on Nov. 28, 2011, in the Korean Intellectual Property Office, the disclosure of which is incorporated herein by reference.

### BACKGROUND OF THE INVENTION

1. Field of the Invention

The present invention relates to an incubating container and a sample injection method therefor and, more particularly, to an incubating container capable of preventing bubble generation within a cell incubating container when a sample is injected thereinto, and a sample injection method therefor.

2. Description of the Related Art

In order to measure the reactions of cells to various medicines, first, an environment for incubating cells needs to be established, and in particular, an incubating container allowing for cells to be easily incubated should be provided.

Also, in order to easily incubate cells, the incubating container is required to be uniformly filled with a culture solution.

However, the incubating container, incubating cells, may be very small. Thus, when bubbles are generated on the bottom of the container in the course of injecting the culture solution into the incubating container, smooth cell culturing may be hindered.

In particular, such bubbles prevent the culture solution from being injected into the container in sufficient quantity. Also, since bubbles occupy a certain volume within the incubating container, cell culture and reaction measurements may not be properly made.

Thus, a cell incubating container capable of preventing bubble generation therein and a sample injection method therefor are required.

### SUMMARY OF THE INVENTION

An aspect of the present invention provides an incubating container capable of preventing bubble generation therein when a sample such as a culture solution is injected thereinto and a sample injection method therefor.

According to an aspect of the present invention, there is provided an incubating container including: a plurality of wells to be filled with a sample through an injection of the sample; and at least one bubble discharge hole formed as a hole penetrating through a bottom of each of the plurality of wells, and discharging, to the outside, bubbles generated when the sample is injected into the plurality of wells.

The bubble discharge hole may have a size allowing only bubbles to be discharged therethrough.

The incubating container may further include a filter disposed within each of the plurality of wells or the bubble discharge hole to only discharge the bubbles to the outside.

Each of the plurality of wells may have a bottom surface and a side wall, and a corner portion in which the bottom surface and the side wall meet may be formed to be curved.

The bubble discharge hole may be formed in the corner portion.

The bubble discharge hole may include two or more bubble discharge holes formed to be spaced apart from each other in a diameter direction of each of the plurality of wells.

Each of the plurality of wells may have an auxiliary recess having an annular shape and formed along the corner portion.

The bubble discharge hole may be disposed within the auxiliary recess.

Each of the plurality of wells may have a linear auxiliary recess connecting the two or more bubble discharge holes disposed to be spaced apart from each other, and formed as a recess in the bottom surface thereof.

In each of the plurality of wells, a depth of a central portion thereof may be greatest and the bubble discharge hole may be formed in the central portion.

The bubble discharge hole may be disposed on a path on which a sample injector allowing for the injection of the sample moves.

According to another aspect of the present invention, there is provided a method of injecting a sample into the incubating container of claim 1, the method including: a first operation of positioning a sample injector above one side wall of each of the plurality of wells and then performing the sample injection; a second operation of continuously injecting the sample while moving the sample injector into the well; and a third operation of stopping the injection of the sample when the sample injector is positioned above the other side wall of each of the plurality of wells.

The method may further include: after performing the third operation, moving the sample injector to a position above another well adjacent to each of the plurality of wells and repeatedly performing the first to third operations.

Each of the plurality of wells may have a bottom surface and a side wall, and the bubble discharge hole may be formed in a corner portion in which the bottom surface and the side wall meet.

In the first operation, the sample may be injected from above the bubble discharge hole.

In the third operation, the injection of the sample may stop above the bubble discharge hole.

In the second operation, the sample injector may move to above the bubble discharge hole.

In the second operation, the sample injector may move in a diameter direction of each of the plurality of wells.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a perspective view schematically showing an incubating container according to an embodiment of the present invention;

FIG. 2 is a partial cross-sectional view, taken along line A-A′ of FIG. 1;

FIG. 3 is a partial plan view of FIG. 2;

FIGS. 4A through 4C are views explaining a sample injection method of an incubating container according to an embodiment of the present invention;

FIG. 5A is a partial plan view of an incubating container according to another embodiment of the present invention;

FIG. 5B is a partial cross-sectional view, taken along line B-B′ of FIG. 5A;

FIG. 6A is a partial plan view of an incubating container according to another embodiment of the present invention;

FIG. 6B is a partial cross-sectional view, taken along line C-C′ of FIG. 5A;

FIG. 7A is a partial plan view of an incubating container according to another embodiment of the present invention;

FIG. 7B is a partial sectional view, taken along line D-D′ of FIG. 7A; and

FIGS. 8A and 8B are partial cross-sectional views of an incubating container according to another embodiment of the present invention.

### DETAILED DESCRIPTION OF THE INVENTION

Prior to the detailed description of the present invention, the configurations described in the embodiments and drawings of the present invention are merely most preferable embodiments but do not represent all of the technical spirit of the present invention. Thus, the present invention should be construed as including all the changes, equivalents, and substitutions included in the spirit and scope of the present invention at the time of filing this application.

The embodiments of the present invention will now be described in detail with reference to accompanying drawings below.

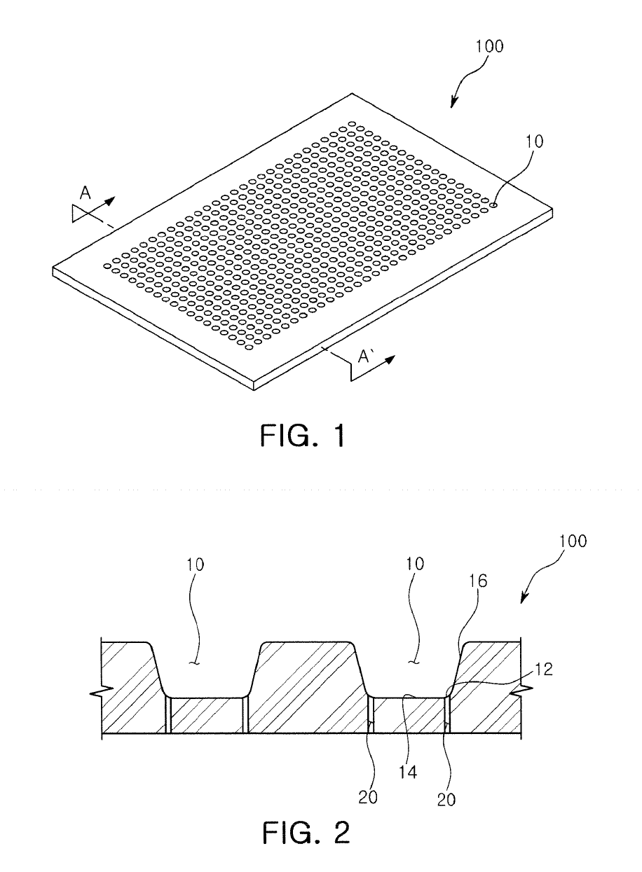

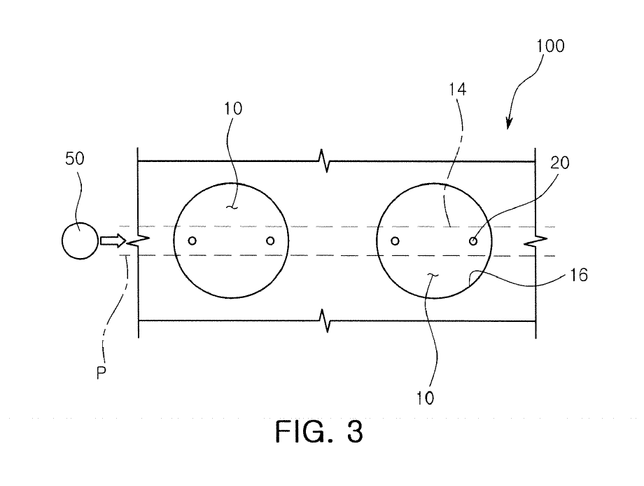

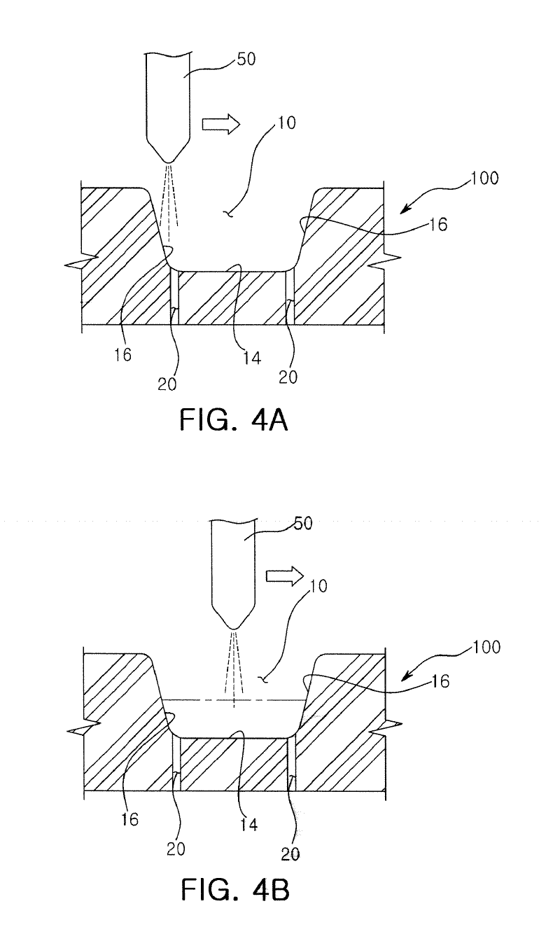

FIG. 1 is a perspective view schematically showing an incubating container according to an embodiment of the present invention; FIG. 2 is a partial cross-sectional view, taken along line A-A′ of FIG. 1; and FIG. 3 is a partial plan view of FIG. 2.

With reference to FIGS. 1 through 3, an incubating container 100 according to an embodiment of the present invention may have a rectangular flat plate shape and includes a plurality of partition areas 10 (hereinafter, referred to as wells) as recesses formed therein. In particular, the incubating container 100 according to the embodiment may be a container for incubating cells.

Thus, a material for a main body of the incubating container 100 is not particularly limited, but preferably, in order to observe the behavior of cells during incubation, a bottom surface 14 of each well 10 may be colorless and transparent or almost colorless and transparent. For example, preferably, the incubating container 100 may be made of plastic, glass, or the like, and plastic such as vinyl chloride, polystyrene, polypropylene, an acrylic material, or the like, may be used therefor.

In the incubating container 100 according to the embodiment, each of the wells 10 has a diameter of about 1 mm and a depth of about 1 mm. An interval between the wells 10 may be about 0.5 mm. However, the present invention is not limited thereto and may be variably modified as necessary.

Also, in the incubating container 100, corner portion 12 in which the bottom surface 14 and side walls 16 of each well 10 meet may be curved. If the corner portions 12 are angular, rather than being curved, bubbles may be easily generated at the corner portions 12 when a sample is injected into the incubating container 100.

Thus, in order to prevent bubble generation at the corner portions 12 of the incubating container 100 according to the embodiment, the corner portions 12 may be curved. Accordingly, the entire inner surface of the wells is smoothly formed without an angular portion.

Meanwhile, in the embodiment, the case in which the respective wells 10 have a circular cross-section is described, but the present invention is not limited thereto and each of the wells 10 maybe provided with an entrance having a polygonal shape such as a quadrangular shape, a hexagonal shape, or the like. Also, in this case, however, all corner portions 12 of the inner surface of each well 10, in which respective surfaces meet, may be curved.

Also, the incubating container 100 according to the embodiment may include at least one bubble discharge hole 20 formed in the bottom surface 14 of each of the wells 10.

The at least one bubble discharge hole 20 is formed to vertically penetrate through a bottom of each well 10. The at least one bubble discharge hole 20 is provided to eliminate bubbles generated from the bottom or the side walls 16 of each well 10 when a sample such as a culture solution, or the like, is injected into the well 10.

Thus, the at least one bubble discharge hole 20 according to the embodiment may be formed as a hole having a size allowing bubbles, i.e., air, to pass therethrough but not allowing a liquid sample injected into the well 10 to pass therethrough.

Here, when the liquid sample is pressurized toward the at least one bubble discharge hole 20, the liquid sample may be discharged through the at least one bubble discharge hole 20. However, the at least one bubble discharge hole 20 according to the embodiment may refer to a hole having a size not allowing a liquid sample to easily pass therethrough only with gravitation due to surface tension of the liquid sample.

The at least one bubble discharge hole 20 may be formed as a through hole having a diameter of, for example, about 10 nm. However, the present invention is not limited thereto.

Due to the presence of the at least one bubble discharge hole 20, when a sample such as a culture solution, or the like, is injected into each well 10, bubbles generated within the well 10 are discharged to the outside of the well 10 through the at least one bubble discharge hole 20 and the interior of the well 10 may be filled only with the sample.

Meanwhile, in order to smoothly discharge bubbles, the at least one bubble discharge hole 20 may be formed in a position in which bubbles are easily generated. To this end, the at least one bubble discharge hole 20 according to the embodiment of the present invention may be disposed on a path (P in FIG. 3) through which a sample injector 50 passes.

In the incubating container 100 according to the embodiment, the sample injector 50 may inject a sample into the respective wells 10 of the incubating container 100 disposed in a lower portion thereof, while moving in an arrow direction shown in FIG. 3 to thereby fill the interior of respective wells 10 with the sample.

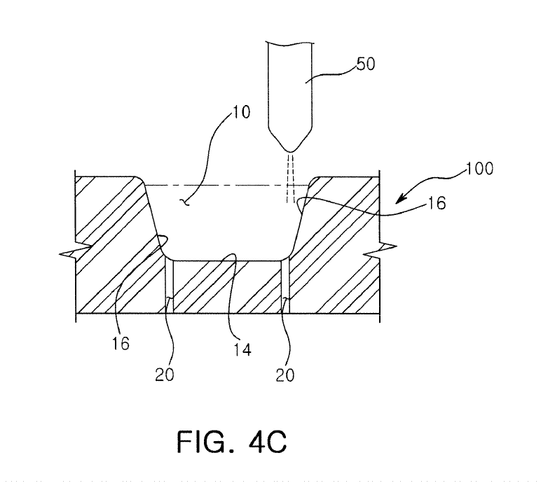

FIGS. 4A through 4C are views explaining a sample injection method of an incubating container according to an embodiment of the present invention.

With reference to FIGS. 4A through 4C, the sample injector 50 according to the embodiment injects a sample, while moving in a diameter direction of the respective wells 10.

First, as shown in FIG. 4A, when the sample injector 50 may be positioned at one side wall 16 of each well 10, the sample injector 50 injects a sample into the well 10. In detail, the sample injector 50 according to the embodiment may be positioned at one corner portion 12 in which the bottom surface 14 and one side wall 16 of each well 10 meet, in particular, the sample injector 50 may be positioned above the air discharge hole 20, and then, inject a sample.

Accordingly, since the sample is injected toward the side wall 16 and the bubble discharge hole 20, rather than toward the bottom surface 14 of the well, impacts when the sample collides with the inner wall of the well 10 can be minimized.

And, as shown in FIG. 4B, the sample injector 50 moves to another bubble discharge hole 20 formed in each well 10. Namely, the sample injector 50 may continuously inject the sample into the well 10, while moving in the diameter direction of the well 10.

Subsequently, as shown in FIG. 4C, when the sample injector 50 may be positioned above the other side wall 16 of the well 10, the sample injector 50 may stop injecting the sample. In detail, when the sample injector 50 may be positioned above the bubble discharge hole 20 formed in the other corner portion of the well 10, the sample injector 50 may stop injecting the sample. Thereafter, the sample injector 50 may move to another adjacent well 10, and repeat the foregoing process in order to inject the sample in the same manner.

When the injecting of the sample starts from one side wall 16 and is completed at the other side wall 16 opposed thereto, the sample may be injected to the corner portions 12 formed to be curved, and thus, impacts generated as the sample collides with the inner wall of the well 10 can be reduced. Accordingly, bubble generation can be minimized.

Meanwhile, through repeated experiments, it was noted that, when the sample was injected while moving the sample injector 50 in the diameter direction of the well 10, bubbles were largely generated from a portion into which the sample was first injected by the sample injector 50 and a portion into which the injection of the sample was stopped after the sample was finally injected.

Thus, as described above, in the incubating container 100 according to the embodiment, the at least one bubble discharge hole 20 may be disposed on the path P on which the sample injector 50 moves. Also, the at least one bubble discharge hole 20 may be disposed in the portions from which bubbles are largely generated, namely, in the portions in which the side walls 16 and the bottom surface 14 meet and through which the sample injector 50 passes.

Meanwhile, the embodiment of the present invention exemplarily illustrates the case in which the at least one bubble discharge hole 20 may include two bubble discharge holes 20, disposed to be spaced apart from each other in the diameter direction of the well 10. However, the present invention is not limited thereto. Namely, two or more bubble discharge holes 20 may be formed, as necessary.

Also, the embodiment of the present invention exemplarily illustrates only the case in which two bubble discharge holes 20 are disposed along both side walls 16 facing each other in the diameter direction of the well 10, but when two or more bubble discharge holes 20 are provided, the bubble discharge holes 20 may be disposed in a row such that they are connected. However, the present invention is not limited thereto and various application thereof, such as a configuration in which a plurality of bubble discharge holes 20 are grouped along respective both side walls 16 of the well 10, facing each other, may be possible.

The incubating container according to the present embodiment configured as described above is not limited to the foregoing embodiment and variably applicable. An incubating container according to embodiments described hereinafter has a similar structure to that of the incubating container (100 in FIG. 1) of the foregoing embodiment, and is different in the structure of the bubble discharge hole. Thus, a detailed description of the same components will be omitted, and the structure of the bubble discharge hole will be described in detail. Also, the same components as those of the foregoing embodiment will be described by using the same reference numerals.

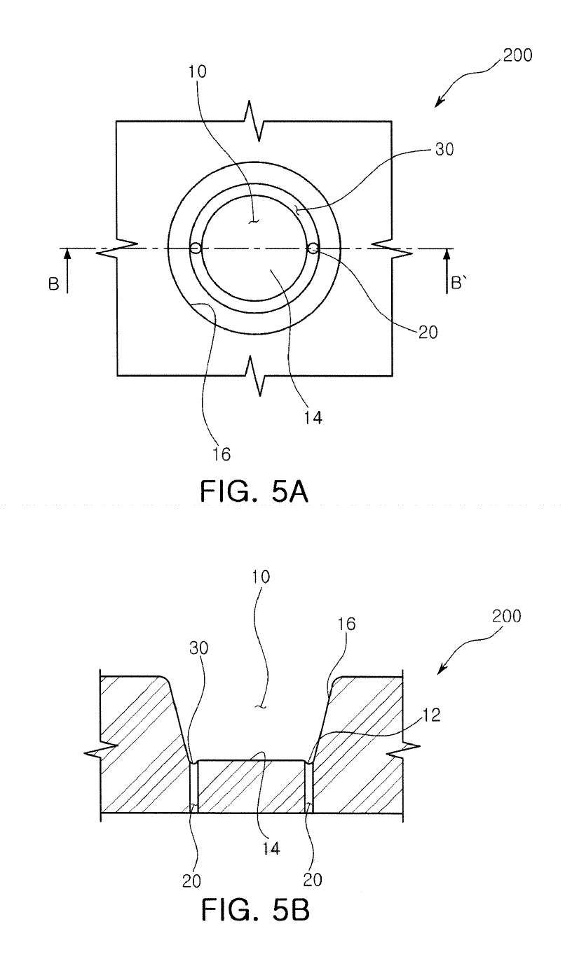

FIG. 5A is a partial plan view of an incubating container according to another embodiment of the present invention. FIG. 5B is a partial cross-sectional view, taken along line B-B′ of FIG. 5A.

With reference to FIGS. 5A and 5B, in an incubating container 200 according to another embodiment of the present invention, an auxiliary recess 30 having an annular shape is formed along the circumference of the bottom surface 14 of each well 10.

The auxiliary recess 30 may be formed along the corner portions in which the bottom surface 14 and the side walls 16 of the well 10 meet. Thus, the at least one bubble discharge hole 20 may be disposed such that an opening at an upper end thereof is opened within the auxiliary recess 30.

Thus, when the auxiliary recess 30 is additionally formed in the well 10, even if bubbles are generated in the corner portions 12, distant from the vicinity of the bubble discharge hole 20, bubbles can be discharged to the outside or bubble generation in the form of being upwardly protruded can be minimized.

Namely, when bubbles are generated in the corner portions 12, distant from the vicinity of the bubble discharge hole 20, bubbles may extend along the auxiliary recess 30 and may be disposed within the auxiliary recess 30.

When bubbles are disposed within the auxiliary recess 30 in this manner, bubbles are disposed to have a small thickness along the bottom surface 14 of the well 10. Thus, even if cells are input into the well 10 afterwards, a phenomenon in which cells are in contact with bubbles can be minimized.

Also, when bubbles extend along the auxiliary recess 30 and meet the bubble discharge hole 20, bubbles can be discharged to the outside through the bubble discharge hole 20.

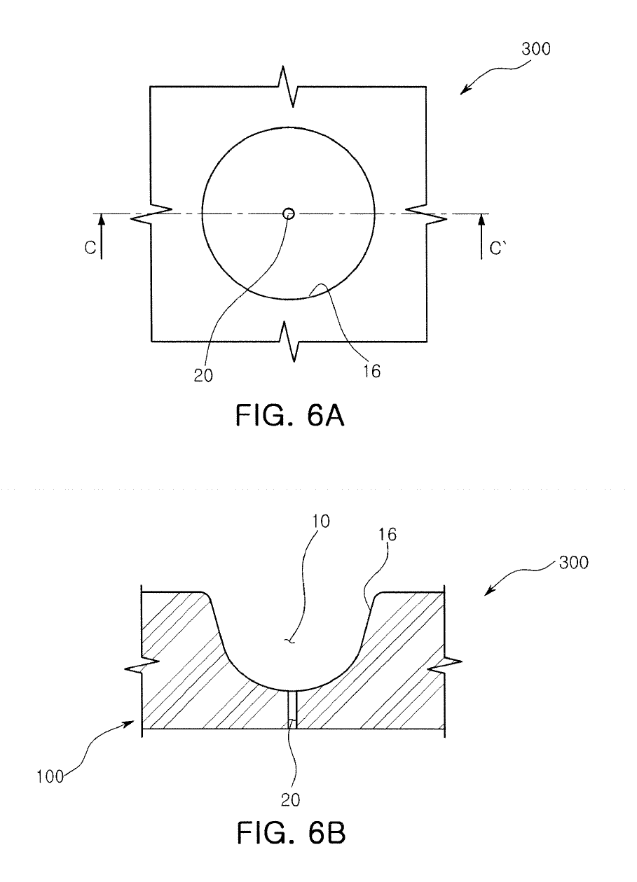

FIG. 6A is a partial plan view of an incubating container according to another embodiment of the present invention. FIG. 6B is a partial cross-sectional view, taken along line C-C′ of FIG. 5A.

With reference to FIGS. 6A and 6B, in an incubating container 300 according to another embodiment, the bubble discharge hole 20 may be formed in the center of the bottom of each well 10.

Also, as for the shape of the well 10, the well 10 does not have the bottom surface 14, which is flat, unlike in the foregoing embodiment but has a curved bottom surface extending from the side wall 16. Namely, in the well 10 according to the embodiment of the present invention, a depth of the central portion thereof is greatest. The bubble discharge hole 20 is formed in the central portion of the well 10.

When the bubble discharge hole 20 is disposed in the central portion of the well 10 in this manner, since the bubble discharge hole 20 may be singularly provided, the incubating container 300 may be easily fabricated.

Meanwhile, in the incubating container 300 according to the present invention, since the bubble discharge hole 20 may be singularly provided, it is required to inject a sample in such a manner that bubbles are largely generated in the formation position of the bubble discharge hole 20.

Thus, a method of injecting a sample into the incubating container 300 according to the embodiment may be performed in a different manner from that of the foregoing method. In detail, the sample injection method according to the embodiment of the present invention may be configured such that the sample injector (50 in FIG. 3) is positioned above the central portion, rather than the side wall 16 of the well 10, namely, above the bubble discharge hole 20, and then, a sample is injected into the well 10. Also, the sample injection method according to the embodiment of the present invention may be configured such that the sample injector 50 may be stationary above the bubble discharge hole 20 during the injection of the sample without a movement thereof, and then, when the injection of the sample is completed, the sample injector 50 may move to another well 10. However, the embodiment of the present invention is not limited thereto.

Meanwhile, in the embodiment, the case in which the bubble discharge hole 20 may be singularly provided, but the embodiment can be variably applicable such as a configuration in which the plurality of bubble discharge holes 20 are grouped in the central portion of the well 10.

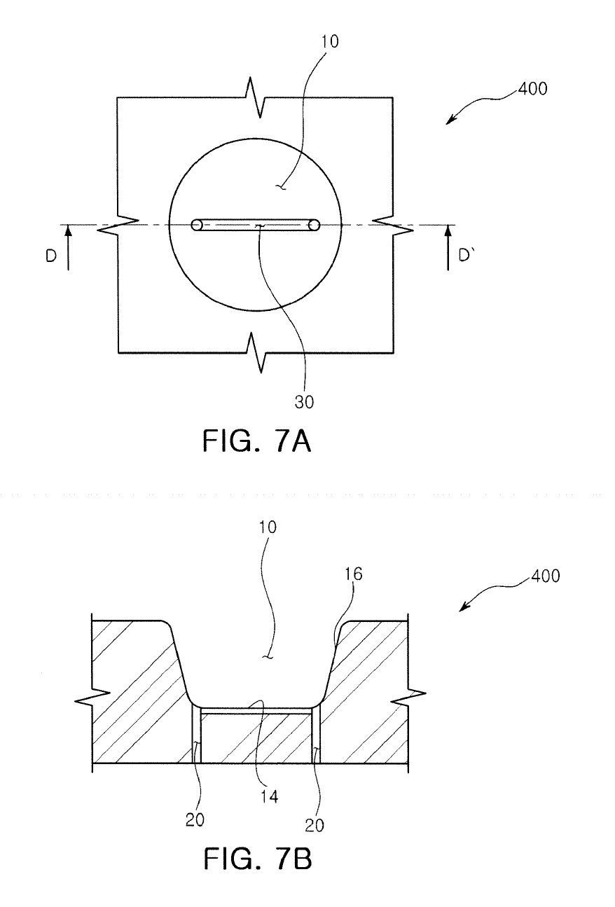

FIG. 7A is a partial plan view of an incubating container according to another embodiment of the present invention. FIG. 7B is a partial sectional view, taken along line D-D′ of FIG. 7A.

With reference to FIGS. 7A and 7B, in an incubating container 400 according to another embodiment, the auxiliary recess 30 connecting two bubble discharge holes 20 may be formed in the bottom surface 14 of the well 10.

Namely, the auxiliary recess 30 is formed in the bottom surface 14 of the well 10 on the path (P in FIG. 3) on which the sample injector (50 in FIG. 3) moves. The auxiliary recess 30 may be formed to have a width similar to the diameter of the bubble discharge holes 20, but the present invention is not limited thereto and the auxiliary recess 30 may have a larger width.

In the case in which the auxiliary recess 30 is additionally formed on the path on which the sample injector 50 moves (indicated by P in FIG. 3) , when bubbles are generated while the sample injector 50 moves, bubbles may extend along the auxiliary recess 30 and disposed within the auxiliary recess 30.

Thus, since bubbles are disposed to have a small thickness along the bottom surface 14 of the well 10, even if cells are input into the well 10 afterwards, a phenomenon in which the cells and bubbles come into contact with each other can be minimized. Also, when bubbles extend along the auxiliary recess 30 and meet the bubble discharge holes 20, bubbles can be discharged to the outside through the bubble discharge holes 20.

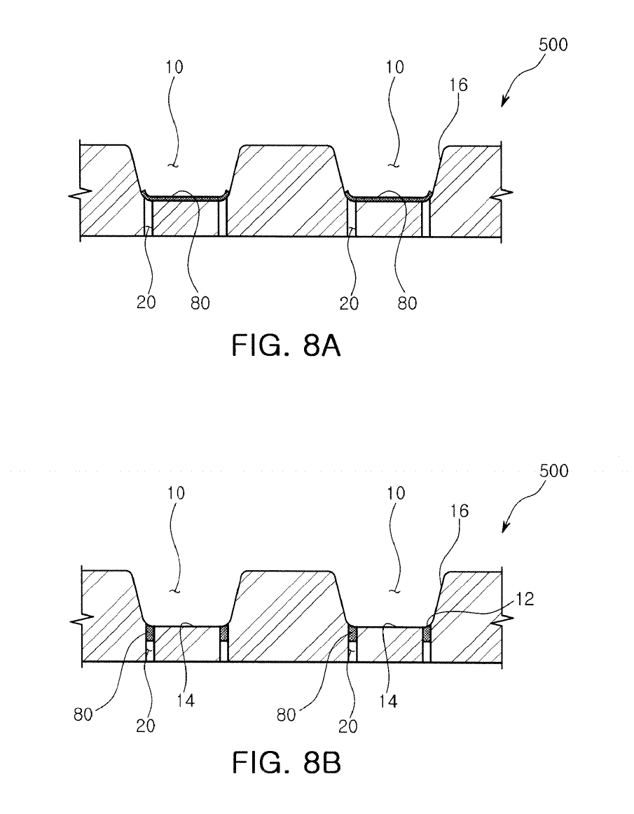

FIGS. 8A and 8B are partial cross-sectional views of an incubating container according to another embodiment of the present invention.

An incubating container 500 according to another embodiment may include a separate filter 80 provided within the well 10.

The filter 80 may be made of a material allowing only a gas to pass therethrough, but not allowing a sample, i.e., a liquid, injected into the well 10 to pass therethrough.

As shown in FIG. 8A, the filter 80 may be disposed on the bottom surface of the well 10 to cover the entrances of the bubble discharge holes 20. The embodiment of the present invention exemplarily illustrates the case in which the filter 80 is singularly provided and covers the entire bottom surface of the well 10, but the present invention is not limited thereto and the present invention can be variably applicable, such as a configuration in which the filter 80 is provided in plural and the plurality of filters 80 cover the respective bubble discharge holes 20, or the like.

In this manner, the sample injected into the well 10 may not penetrate through the filter 80. Thus, the sample is accommodated only within the well 10 by the filter 80, and may not be leaked to the outside through the bubble discharge holes 20.

Thus, when the filter 80 is provided according to the embodiment of the present invention, the incubating container 500 may be configured regardless of the size (i.e., diameter) of the bubble discharge holes 20. Namely, even if the bubble discharge holes 20 are formed to have a size allowing the sample to easily flow thereinto, since the sample may not be introduced into the bubble discharge holes 20 by the filter 80, the bubble discharge holes 20 may have various sizes.

Meanwhile, the filter 80 according to the embodiment of the present invention is not limited to the foregoing embodiment and variably applicable. For example, as shown in FIG. 8B, the filter 80 may be inserted into the bubble discharge holes 20.

As set forth above, in the incubating container according to the embodiments of the invention, the corner portions in which the bottom surface and the side walls of the well meet are formed to be curved in order to prevent bubble generation in the corner portions.

Thus, since the entirety of the interior of the well is smoothly formed without an angular portion, bubble generation in the angular portion during the injection of the sample can be prevented.

Also, in the method of injecting the sample into the culture solution, the injection of the sample starts from one side wall of the well and is continuously maintained through the movement to the other side wall opposed thereto, and then, the injection is completed as the sample arrives at the other side wall. Thus, since impacts generated between the sample and the well may be minimized, bubble generation may be minimized.

Also, the incubating container according to the embodiment of the present invention includes at least one bubble discharge hole. Thus, when a sample, such as a culture solution, or the like, is injected, bubbles generated within the well may be discharged to the outside of the well through the at least one bubble discharge hole and the interior of the well may be filled only with the sample.

In addition, the at least one bubble discharge hole according to the embodiment of the present invention may be disposed in a path in which the sample is injected, rather than being disposed in an arbitrary position. Namely, the at least one bubble discharge hole may be selectively disposed in a position in which bubbles are largely generated. Thus, bubbles generated within the well can be effectively discharged.

Also, although not shown, bubbles in the incubating container according to the embodiment of the present invention may be discharged by using a separate suction device.

For example, a vacuum suction device may be provided and the incubating container according to the embodiment of the present invention may be disposed on a suction unit of the vacuum suction device. In this case, the incubating container may be disposed such that a lower surface thereof is tightly attached to the suction unit.

In this case, bubbles generated within the well of the incubating container can be easily discharged to the outside, i.e., discharged downwardly of the incubating container through the bubble discharge hole by vacuum suction of the vacuum suction device.

Meanwhile, the incubating container according to the present invention is not limited to the foregoing embodiments and maybe variably modified by a person skilled in the art within the scope of the technical concept of the present invention.

For example, the foregoing embodiments exemplarily illustrate the case of using the incubating container having a rectangular shape, but the present invention is not limited thereto. Namely, the present invention can be variably applicable and the incubating container may be configured to have a circular shape, an oval shape, a polygonal shape, or the like.

Also, in the foregoing embodiments, the incubating container for incubating cells has been described as an example, but the present invention is not limited thereto. Namely, the present invention can be variably applicable to a device for medicating cells, a device for detecting a bio-material, and the like, so long as it is a device for allowing cells, microorganisms, or the like, to react to a liquid sample.

While the present invention has been shown and described in connection with the embodiments, it will be apparent to those skilled in the art that modifications and variations can be made without departing from the spirit and scope of the invention as defined by the appended claims.

What is claimed is:

1. An incubating container comprising:
a plurality of wells to be filled with a sample through an injection of the sample; and
at least one bubble discharge hole formed as a hole penetrating through a bottom of each of the plurality of wells, and discharging, to the outside, bubbles generated when the sample is injected into the plurality of wells.

2. The incubating container of claim 1, wherein the bubble discharge hole has a size allowing only bubbles to be discharged therethrough.

3. The incubating container of claim 1, further comprising a filter disposed within each of the plurality of wells or the bubble discharge hole to only discharge the bubbles to the outside.

4. The incubating container of claim 1, wherein each of the plurality of wells has a bottom surface and a side wall, and a corner portion in which the bottom surface and the side wall meet is formed to be curved.

5. The incubating container of claim 4, wherein the bubble discharge hole is formed in the corner portion.

6. The incubating container of claim 5, wherein the bubble discharge hole includes two or more bubble discharge holes formed to be spaced apart from each other in a diameter direction of each of the plurality of wells.

7. The incubating container of claim 4, wherein each of the plurality of wells has an auxiliary recess having an annular shape and formed along the corner portion.

8. The incubating container of claim 7, wherein the bubble discharge hole is disposed within the auxiliary recess.

9. The incubating container of claim 6, wherein each of the plurality of wells has a linear auxiliary recess connecting the two or more bubble discharge holes disposed to be spaced apart from each other, and formed as a recess in the bottom surface thereof.

10. The incubating container of claim 1, wherein in each of the plurality of wells, a depth of a central portion thereof is greatest and the bubble discharge hole is formed in the central portion.

11. The incubating container of claim 1, wherein the bubble discharge hole is disposed on a path on which a sample injector allowing for the injection of the sample moves.

12. A method of injecting a sample into the incubating container of claim 1, the method comprising:
a first operation of positioning a sample injector above one side wall of each of the plurality of wells and then performing the sample injection;
a second operation of continuously injecting the sample while moving the sample injector into the well; and
a third operation of stopping the injection of the sample when the sample injector is positioned above the other side wall of each of the plurality of wells.

13. The method of claim 12, further comprising, after performing the third operation, moving the sample injector to a position above another well adjacent to each of the plurality of wells and repeatedly performing the first to third operations.

14. The method of claim 12, wherein each of the plurality of wells has a bottom surface and a side wall, and the bubble discharge hole is formed in a corner portion in which the bottom surface and the side wall meet.

15. The method of claim 14, wherein in the first operation, the sample is injected from above the bubble discharge hole.

16. The method of claim 14, wherein in the third operation, the injection of the sample stops above the bubble discharge hole.

17. The method of claim 14, wherein in the second operation, the sample injector moves to above the bubble discharge hole.

18. The method of claim 12, wherein in the second operation, the sample injector moves in a diameter direction of each of the plurality of wells.
